(19) United States
(12) Patent Application Publication
(10) Pub. No.: US 2022/0395238 A1
(43) Pub. Date: Dec. 15, 2022

(54) REAL-TIME FITNESS COACHING SYSTEM USING SMART HEALTH SENSOR

(71) Applicant: RC-Tech Co.,Ltd., Yongin-si (KR)

(72) Inventor: Tae hwa LIM, Yongin-si (KR)

(21) Appl. No.: 17/806,682

(22) Filed: Jun. 13, 2022

(30) Foreign Application Priority Data
Jun. 11, 2021 (KR) 10-2021-0076164

(51) Int. Cl. A61B 5/00; A61B 5/11; G16H 20/30

(52) U.S. Cl. CPC A61B 5/7455; A61B 5/11; A61B 5/7405; G16H 20/30; A61B 5/6806; A61B 2562/08

(57) ABSTRACT

Provided is a real-time fitness coaching system using a smart health sensor. The real-time fitness coaching system includes smart fitness equipment including a reference position sensor disposed at a preset position, a smart wearable band which includes a motion position sensor and is attached to a part of an individual's body, and a server connected to the smart fitness equipment and the smart wearable band via a network, wherein the server receives reference position information of the reference position sensor from the smart fitness equipment, receives motion position information of the motion position sensor from the smart wearable band, generates fitness correction information on the basis of a relative value of the motion position information with respect to the reference position information, and provides the fitness correction information to a user terminal connected thereto via a network.

### CROSS-REFERENCE TO RELATED APPLICATION

This application claims priority under 35 U.S.C § 119 to Korean Patent Application No. 10-2021-0076164, filed in the Korean Intellectual Property Office on Jun. 11, 2021, the disclosure of which is incorporated herein by reference in its entirety.

### BACKGROUND

### 1. Field of the Invention

The present invention relates to a fitness coaching system, and more particularly, to a real-time fitness coaching system using a smart health sensor, which is capable of providing fitness correction information in real time by acquiring individual motion information using a smart health sensor.

### 2. Discussion of Related Art

Recently, as interest in health has increased, many people have been visiting fitness clubs to exercise. However, in the case of beginners, they are receiving personal training (PT) from health trainers because they lack the knowledge to exercise correctly for each piece of exercise equipment.

However, it is costly for individuals to receive private PT lessons from health trainers, and thus a significant number of people exercise without private PT lessons. Basically, there are many cases where a health trainer informs people about exercise methods, but, there are problems in that individuals cannot easily receive detailed correction, have difficulty maximizing an effect of the exercise, and can experience side effects after exercising for a long period of time with the wrong posture.

In order to solve the above problems, in Korean Laid-open Patent Application No. 10-2018-0088960, an exercise correction system using smart gloves is disclosed. The system is a system for providing information on exercise correction according to pressure values applied to both hands during exercise using smart gloves having a pressure sensor. The above exercise correction system has a limitation in that only correction information according to a difference in pressure values applied to both hands is presented and information on posture correction suitable for various pieces of exercise equipment is not provided.

In addition, in Korean Laid-open Patent Application No. 10-2021-0041669, a smart health glove system including a pressure sensor and a gyro sensor is disclosed. In the smart health glove system, a configuration in which a pressure value applied to exercise equipment is measured using a pressure sensor and a posture is measured using a gyro sensor in order to suggest posture correction method is disclosed. However, in the smart health glove system, only a concept of presenting a method of measuring and correcting a posture using a gyro sensor is disclosed, and a method of measuring and correcting a posture is not disclosed. Further, in the smart health glove system, a method of measuring a posture for each of various pieces of exercise equipment and providing customized correction information is not disclosed.

### DOCUMENT OF RELATED ART

### Patent Document

(Patent Document 1) Korean Laid-open Patent Application No. 10-2018-0088960 (Published on Aug. 8, 2018)

(Patent Document 2) Korean Laid-open Patent Application No. 10-2021-0041669 (Published on Apr. 16, 2021)

### SUMMARY OF THE INVENTION

The present disclosure is directed to solve the above problem, and specifically, is directed to providing a real-time fitness coaching system using a smart health sensor, which is capable of providing fitness correction information in real time by acquiring individual exercise motion information for each of various pieces of exercise equipment using a smart health sensor.

Objects of the present disclosure are not limited to the above-described object and other objects that are not described may be clearly understood by those skilled in the art from the following descriptions.

According to an aspect of the present disclosure, there is provided a real-time fitness coaching system using a smart health sensor, including smart fitness equipment including a reference position sensor disposed at a preset position, a smart wearable band which includes a motion position sensor and is attached to a part of an individual's body, and a server connected to the smart fitness equipment and the smart wearable band via a network, wherein the server receives reference position information of the reference position sensor from the smart fitness equipment, receives motion position information of the motion position sensor from the smart wearable band, generates fitness correction information on the basis of a relative value of the motion position information with respect to the reference position information, and provides the fitness correction information to a user terminal connected thereto via a network.

The reference position sensor may include a first reference position sensor and a second reference position sensor which are disposed in the smart fitness equipment to be symmetrical with each other, and the smart wearable band may include a first smart wearable band and a second smart wearable band which are disposed on the part of the individual's body to be symmetrical with each other, wherein the server may generate the fitness correction information on the basis of a relative value of first motion position information received from the first smart wearable band with respect to first reference position information received from the first reference position sensor and a relative value of second motion position information received from the second smart wearable band with respect to second reference position information received from the second reference position sensor.

The reference position sensor may further include a third reference position sensor, wherein the server may receive third reference position information from the third reference position sensor, compare a first area calculated from the first reference position information, the third reference position information, and the first motion position information to a second area calculated from the second reference position information, the third reference position information, and the second motion position information, and generate the fitness correction information.

The smart fitness equipment may further include an identification (ID) module including type information of fitness equipment, wherein the server may include a database for a plurality of pieces of fitness equipment for providing the fitness correction information, call the database corresponding to the smart fitness equipment according to the type information of the fitness equipment received from the ID module, and generate the fitness correction information.

The smart wearable band may further include a pressure sensor, wherein the server may generate the fitness correction information on the basis of pressure information received from the pressure sensor.

The smart wearable band may further include a feedback module that provides feedback to a user in a preset manner, wherein the smart wearable band may receive the fitness correction information from the user terminal, and the feedback module may provide the feedback to the user on the basis of the fitness correction information.

The feedback module may be a vibration module, and the feedback may be a vibration pattern generated by the vibration module.

The feedback module may be a speaker module, and the feedback may be a sound pattern or a guidance message generated by the speaker module.

The server may provide a fitness motion to the user terminal as virtual image information on the basis of the reference position information and the motion position information.

The server may add a correction trace corresponding to the fitness correction information to the virtual image information and provide the virtual image information to the user terminal.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a schematic diagram illustrating a network in which a real-time fitness coaching system using a smart health sensor according to an embodiment of the present disclosure is applied;

FIG. 2 is a detailed configuration diagram of first smart fitness equipment of FIG. 1;

FIG. 3 is a detailed configuration diagram of a smart wearable band of FIG. 1;

FIG. 4 is a detailed configuration diagram of a server of FIG. 1;

FIG. 5 is a view illustrating an example in which a user wearing smart wearable bands exercises using the first smart fitness equipment;

FIG. 6 is a conceptual diagram for describing an example of a method of generating, by the server of FIG. 1, fitness correction information using reference position information and motion position information; and

FIG. 7 is a schematic flowchart for describing an operation of the server of FIG. 1.

### DETAILED DESCRIPTION OF EXEMPLARY EMBODIMENTS

Advantages and features of the present disclosure and methods of achieving the same will be clearly understood with reference to the accompanying drawings and embodiments described below in detail. However, the present disclosure is not limited to the embodiments to be disclosed below but may be implemented in various different forms. The embodiments are provided in order to fully explain the present embodiments and fully explain the scope of the present disclosure for those skilled in the art. The scope of the present disclosure is only defined by the appended claims. Like reference numerals refer to like elements throughout the specification.

Hereinafter, a real-time fitness coaching system using a smart health sensor according to embodiments of the present disclosure will be described with reference to the accompanying drawings.

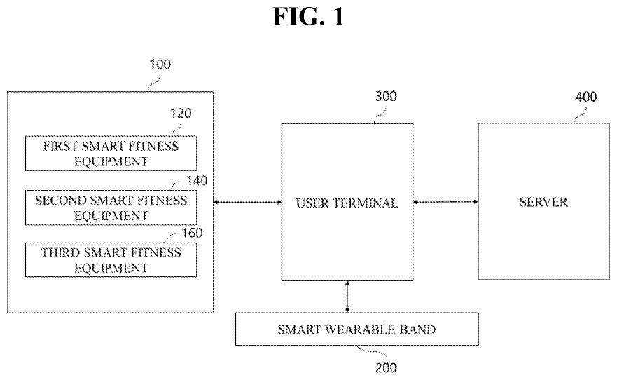

FIG. 1 is a schematic diagram illustrating a network in which a real-time fitness coaching system using a smart health sensor according to an embodiment of the present disclosure is applied.

Referring to FIG. 1, the fitness coaching system using the smart health sensor according to the embodiment of the present disclosure includes smart fitness equipment 100, a smart wearable band 200, and a server 400.

The smart fitness equipment 100, the smart wearable band 200, and the server 400 may be connected to a user terminal 300 via a wired or wireless network.

In this case, the user terminal 300 may be a smartphone, a personal digital assistant (PDA), a tablet personal computer (PC), a notebook computer, etc. of a user who exercises.

The smart fitness equipment 100 may include first smart fitness equipment 120, second smart fitness equipment 140, and third smart fitness equipment 160. This configuration is only an example, and the smart fitness equipment 100 may be composed of one piece of smart fitness equipment, two pieces of smart fitness equipment, or four or more pieces of smart fitness equipment.

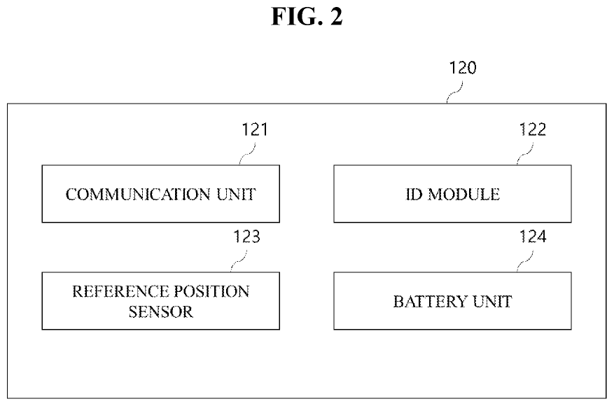

A smart health sensor is attached to the smart fitness equipment 100, and a detailed configuration thereof will be described below with reference to FIG. 2.

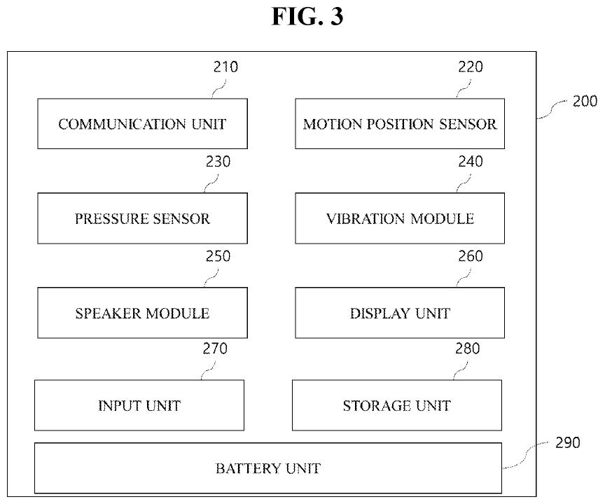

The smart wearable band 200 is a device that the user attaches to a specific part of the user's body during exercise. A smart health sensor is also attached to the smart wearable band 200, and a detailed configuration thereof will be described below with reference to FIG. 3.

The smart wearable band 200 may be configured in a band type as the name suggests, but the present is not limited thereto, and the smart wearable band 200 may be configured in various types such as fitness gloves and the like.

The server 400 is configured in the form of a computer for a server and is directly or indirectly connected to the smart fitness equipment 100, the smart wearable band 200 and the user terminal 300 via the network.

FIG. 1 illustrates a configuration in which the smart fitness equipment 100 and the smart wearable band 200 are connected to the user terminal 300 via the network, and the user terminal 300 is connected to the server 400 via the network.

However, in the real-time fitness coaching system using the smart health sensor according to the embodiment of the present disclosure, unlike the configuration of FIG. 1, the server 400 may be configured in another form such as being directly connected to the smart fitness equipment 100, the smart wearable band 200, and the user terminal 300 via the network, or the like.

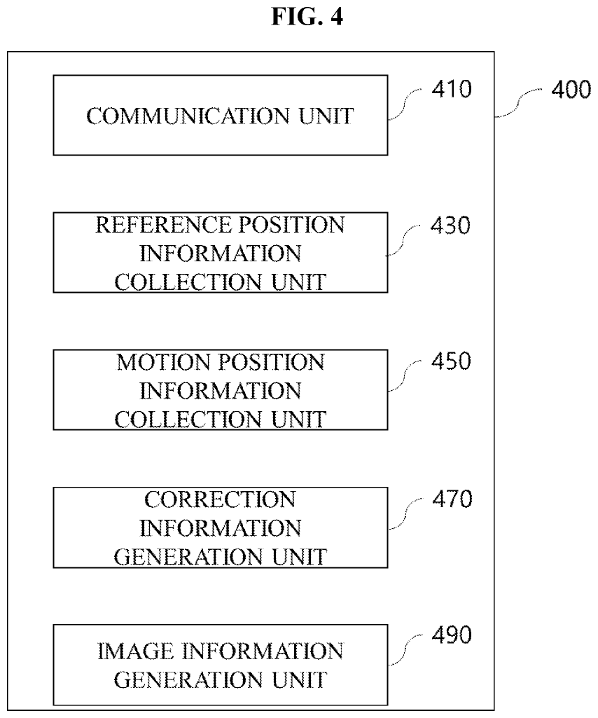

A detailed configuration and operation of the server 400 will be described below with reference to FIG. 4.

FIG. 2 is a detailed configuration diagram of the first smart fitness equipment of FIG. 1.

Referring to FIG. 2, the first smart fitness equipment 120 may include a communication unit 121, an identification (ID) module 122, a reference position sensor 123, and a battery unit 124.

Although not illustrated in FIG. 2, the second smart fitness equipment 140 and the third smart fitness equipment 160 of FIG. 1 may be configured in the same manner as the first smart fitness equipment 120.

The communication unit 121 serves to transmit or receive data to or from an external device. For example, the communication unit 121 may transmit or receive data to or from the user terminal 300.

The communication unit 121 may be configured as a communication module capable of communicating using Wi-Fi or Bluetooth. However, the present disclosure is not limited thereto, and the communication unit 121 may be configured as a communication module using another type of communication method such as communication using a third-, fourth-, or fifth-generation mobile communication network, satellite communication, or the like.

The ID module 122 may store type information of the first smart fitness equipment 120 and transmit the type information of the first smart fitness equipment 120 to the user terminal 300 through the communication unit 121.

The reference position sensor 123 is a sensor disposed to measure three-dimensional (3D) position information. The reference position sensor 123 may be composed of a Global Positioning System (GPS) sensor, but the present disclosure is not limited thereto, and the reference position sensor 123 may be composed of any one of various sensors such as an ultrasonic sensor, an optical sensor, and the like.

The battery unit 124 may serve to supply power to the communication unit 121, the ID module 122, and the reference position sensor 123 and may be configured as an attachable/detachable battery or a rechargeable battery.

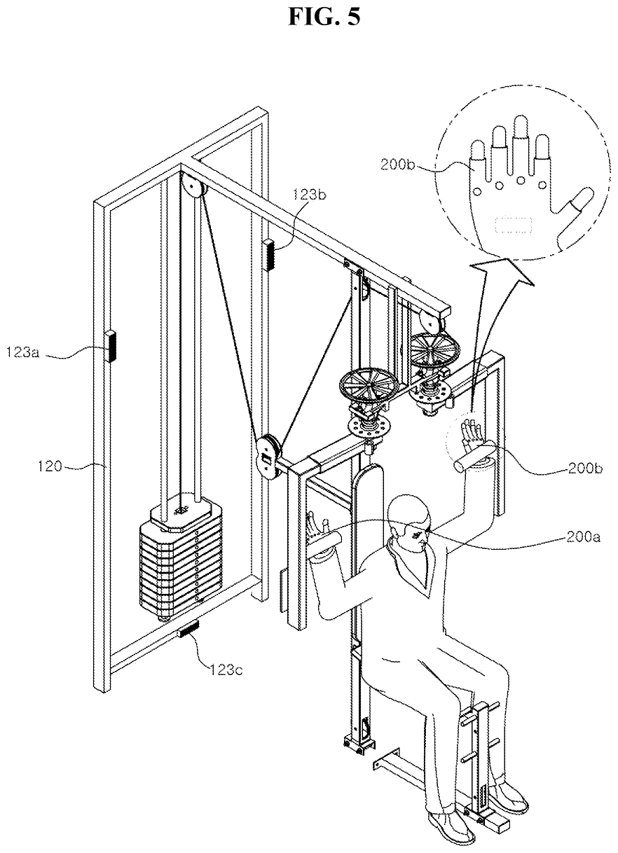

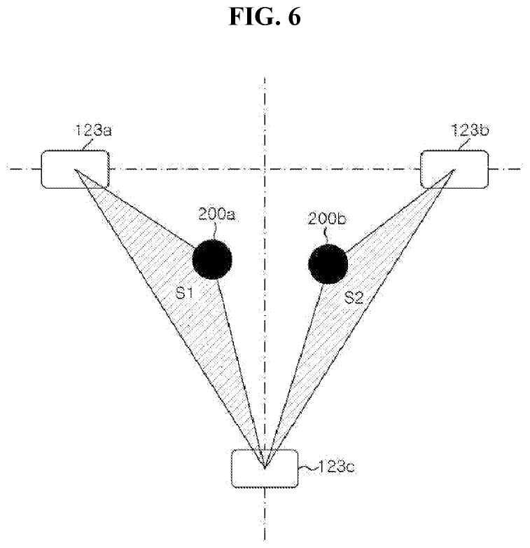

The reference position sensor 123 may be provided with one reference position sensor 123 or may be provided with a plurality of reference position sensors 123 such as three or more reference position sensors 123. An example in which the reference position sensor 123 is provided with three reference position sensors 123 will be described below with reference to FIGS. 5 and 6.

FIG. 3 is a detailed configuration diagram of the smart wearable band of FIG. 1.

Referring to FIG. 3, the smart wearable band 200 of the real-time fitness coaching system using the smart health sensor according to the embodiment of the present disclosure may include a communication unit 210, a motion position sensor 220, a pressure sensor 230, a vibration module 240, a speaker module 250, a display unit 260, an input unit 270, a storage unit 280, and a battery unit 290.

The communication unit 210 serves to transmit or receive data to or from an external device. For example, the communication unit 210 may transmit or receive data to or from the user terminal 300.

The communication unit 210 may be configured as a communication module capable of communicating using Wi-Fi or Bluetooth. However the present disclosure is not limited thereto, and the communication unit 210 may be configured as a communication module using another type of communication method such as communication using a third-, fourth-, or fifth-generation mobile communication network, satellite communication, or the like.

The motion position sensor 220 serves to measure position information or motion information on the smart wearable band 200. The motion position sensor 220 may include any one of a GPS sensor, a gyro sensor, an acceleration sensor, a motion sensor, an ultrasonic sensor, and an optical sensor, or a combination thereof.

The pressure sensor 230 serves to measure pressure applied to the smart wearable band 200. The pressure sensor 230 may be configured as a single sensor module to measure pressure of any one part or may be configured as a plurality of sensor modules to measure pressure of a plurality of parts.

The vibration module 240 serves to generate vibrations in the form of a preset vibration pattern at a specific position of the smart wearable band 200.

The vibration module 240 may be configured to allow a preset algorithm to generate a vibration pattern according to fitness correction information which is provided by the server 400 and transmitted through the user terminal 300.

For example, the vibration module 240 may guide fitness correction by generating vibrations with large intensity when it is determined that the applied pressure is low, and generating vibrations with small intensity when it is determined that the applied pressure is too high.

Further, when it is determined that motion position information is incorrect or a motion speed is high, the vibration module 240 may generate a vibration pattern instructing motion in a specific direction or a vibration pattern guiding a slow motion speed.

The above is only an example of a vibration pattern, and various types of vibration intensity and patterns may be generated as long as they can guide fitness correction.

The speaker module 250 may be configured to allow a preset algorithm to generate a sound pattern or a guidance message according to the fitness correction information which is provided by the server 400 and transmitted through the user terminal 300.

The display unit 260 serves to visually display various types of status indications or correction information of the smart wearable band 200.

For example, the display unit 260 may include a light-emitting diode (LED) lamp, and may display information such as a connection state of the communication unit 210, a battery state of the battery unit 290, and the like through a color or blinking operation.

Further, the display unit 260 may include an LED display unit to display the fitness correction information in text or the like and provide the fitness correction information to the user.

The input unit 270 may be configured in the form of a button or the like to receive a power-on/off operation and environment settings of the smart wearable band 200 as inputs from the user.

The storage unit 280 serves to store various types of environment setting information of the smart wearable band 200, measurement values of the motion position sensor 220 and the pressure sensor 230, and the like.

The battery unit 290 may serve to supply power to the communication unit 210, the motion position sensor 220, the pressure sensor 230, the vibration module 240, the speaker module 250, the display unit 260, the input unit 270, and the storage unit 280, and may be configured as an attachable/detachable battery or a rechargeable battery.

FIG. 4 is a detailed configuration diagram of the server of FIG. 1.

Referring to FIG. 4, the server 400 of the real-time fitness coaching system using the smart health sensor according to the embodiment of the present disclosure may include a communication unit 410, a reference position information collection unit 430, a motion position information collection unit 450, a correction information generation unit 470, and an image information generation unit 490.

The communication unit 410 serves to transmit or receive data to or from an external device. For example, the communication unit 210 may transmit or receive data to or from the user terminal 300.

The communication unit 410 may be configured as a communication module capable of communicating using Wi-Fi or Bluetooth. However, the present disclosure is not limited thereto, and the communication unit 410 may be configured as a communication module using another type of communication method such as communication using a third-, fourth-, or fifth-generation mobile communication network, satellite communication, or the like.

The reference position information collection unit 430 serves to collect reference position information from the reference position sensor 123 of the smart fitness equipment 100.

The motion position information collection unit 450 serves to collect motion position information and pressure information from the motion position sensor 220 and the pressure sensor 230 of the smart wearable band 200.

The correction information generation unit 470 serves to generate fitness correction information using a preset algorithm on the basis of sensor values measured by the reference position information collection unit 430 and the motion position information collection unit 450.

The method of generating, by the correction information generation unit 470, the fitness correction information may be configured in various ways, which will be described below with reference to FIGS. 5 and 6.

The image information generation unit 490 serves to generate virtual image information on the user's fitness motion on the basis of the sensor values measured by the reference position information collection unit 430 and the motion position information collection unit 450.

The virtual image information may include a correction trace corresponding to the fitness correction information.

FIG. 5 is a view illustrating an example in which a user wearing smart wearable bands exercises using the first smart fitness equipment.

FIG. 5 illustrates a case in which the first smart fitness equipment 120 is a butterfly mechanism for chest exercise, and an example in which three of the reference position sensors 123 are provided.

The reference position sensor 123 includes a first reference position sensor 123a, a second reference position sensor 123b, and a third reference position sensor 123c.

The first reference position sensor 123a and the second reference position sensor 123b are symmetrically disposed on both sides of a frame supporting a weight of the first smart fitness equipment 120, and the third reference position sensor 123c is disposed on a center of a lower side of the frame supporting the weight of the first smart fitness equipment 120.

The user exercises while wearing a first smart wearable band 200a and a second smart wearable band 200b made in the form of gloves on both hands.

FIG. 6 is a conceptual diagram for describing an example of a method of generating, by the server of FIG. 1, fitness correction information using reference position information and motion position information.

The conceptual diagram of FIG. 6 illustrates positions of the first smart wearable band 200a and the second smart wearable band 200b and positions of the first reference position sensor 123a, the second reference position sensor 123b, and the third reference position sensor 123c at a specific point in time when the user performs a chest exercise using the first smart fitness equipment 120 of FIG. 5.

It can be confirmed that, since the user applies more force to the user's right hand, the first smart wearable band 200a is disposed closer to a vertical reference line.

The correction information generation unit 470 calculates an area of a triangle S1 on the basis of information on the positions of the first reference position sensor 123a and the third reference position sensor 123c and information on the position of the first smart wearable band 200a.

Further, the correction information generation unit 370 calculates an area of a triangle S2 on the basis of information on the positions of the second reference position sensor 123b and the third reference position sensor 123c and information on the position of the second smart wearable band 200b.

Since the area of the triangle S1 is greater than the area of the triangle S2, it is determined that the user is exercising asymmetrically with a wrong posture, and thus the correction information generation unit 470 may generate fitness correction information by subtracting more force from the first smart wearable band 200a or adding force to the second smart wearable band 200b.

In this case, as described above, the fitness correction information may be directly provided through the vibration module 240, the speaker module 250, and the display unit 260 of the smart wearable band 200 or may be provided through an application or program driven on the user terminal 300.

The application or program is provided by the server 400, and may be driven in the form of a program downloadable to the user terminal 300 or driven by the server 400 on the Web.

Although the method of generating, by the correction information generation unit 470, the fitness correction information by calculating the areas of the triangles is shown in FIG. 6, this is only an example, and it is possible to generate the fitness correction information in various other ways according to the number of the reference position sensors 123 and the like.

For example, the server 400 may include a database such as optimal motion trace information or the like for each type of smart fitness equipment 100 in advance, and an algorithm for providing fitness correction information for each piece of smart fitness equipment 100 may be built in the server 400.

For example, the correction information generation unit 470 may collect only motion position information of the smart wearable band 200 without reference position information and apply the motion position information to the database and algorithm to provide fitness correction information.

However, in order to increase accuracy, the correction information generation unit 470 may provide the fitness correction information in further consideration of one or more pieces of reference position information.

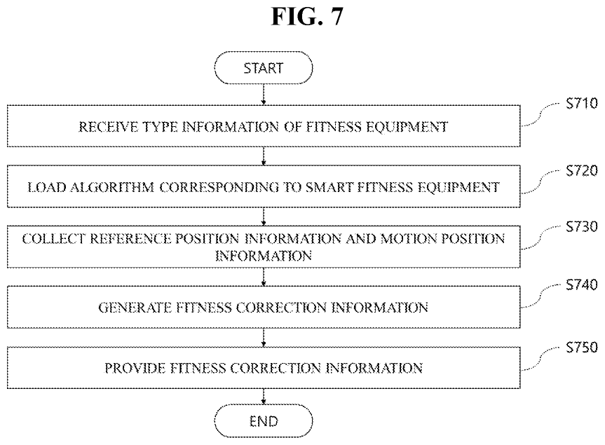

FIG. 7 is a schematic flowchart for describing an operation of the server of FIG. 1.

When a user wears a smart wearable band 200 on a part of the user's body and is positioned on desired smart fitness equipment 100, the server 400 automatically determines that the user is positioned on the specific smart fitness equipment 100 on the basis of sensor values of a reference position sensor 123 and a motion position sensor 220, and the server 400 receives type information of the fitness equipment from an ID module 122 of the smart fitness equipment 100 (S710).

In this case, the fact that the user is positioned on the specific smart fitness equipment 100 may be determined using the smart fitness equipment 100 and the smart wearable band 200 communicating with each other or a proximity sensor or the like in addition to the above method.

When the server 400 receives the type information of the fitness equipment, the server 400 calls an algorithm corresponding to the type of the corresponding smart fitness equipment 100 on the basis of the received type information (S720).

When the user starts to exercise using the smart fitness equipment 100, the server 400 collects reference position information and motion position information from the smart fitness equipment 100 and the smart wearable band 200 in real time (S730).

The server 400 may generate fitness correction information on the basis of a database and algorithm built in advance based on the collected data (S740), and the server 400 may provide the generated fitness correction information to a user terminal (S750).

The real-time fitness coaching system using the smart health sensor according to the embodiment of the present disclosure may accurately provide fitness correction information corresponding to each piece of fitness equipment in real time with the above configuration.

The real-time fitness coaching system using the smart health sensor according to the embodiment of the present disclosure can provide fitness correction information in real time by acquiring individual exercise motion information for each of various pieces of exercise equipment using a smart health sensor.

Effects of the present disclosure are not limited to the above-described effect and more various effects are included in the specification.

The above description of the present disclosure is only exemplary, and it will be understood by those skilled in the art that various modifications can be made without departing from the scope of the present disclosure and without changing essential features. Therefore, the above-described embodiments should be considered in a descriptive sense only and not for purposes of limitation. Accordingly, the scope of the present disclosure is not limited by the embodiments. The scope of the present disclosure is defined by the appended claims and encompasses all modifications and equivalents that fall within the scope of the appended claims.

What is claimed is:

1. A real-time fitness coaching system using a smart health sensor, comprising:
smart fitness equipment including a reference position sensor disposed at a preset position;
a smart wearable band which includes a motion position sensor and is attached to a part of an individual's body; and
a server connected to the smart fitness equipment and the smart wearable band via a network,
wherein the server receives reference position information of the reference position sensor from the smart fitness equipment, receives motion position information of the motion position sensor from the smart wearable band, generates fitness correction information on the basis of a relative value of the motion position information with respect to the reference position information, and provides the fitness correction information to a user terminal connected thereto via a network.

2. The real-time fitness coaching system of claim 1, wherein:
the reference position sensor includes a first reference position sensor and a second reference position sensor which are disposed in the smart fitness equipment to be symmetrical with each other;
the smart wearable band includes a first smart wearable band and a second smart wearable band which are disposed on the part of the individual's body to be symmetrical with each other; and
the server generates the fitness correction information on the basis of a relative value of first motion position information received from the first smart wearable band with respect to first reference position information received from the first reference position sensor and a relative value of second motion position information received from the second smart wearable band with respect to second reference position information received from the second reference position sensor.

3. The real-time fitness coaching system of claim 2, wherein the reference position sensor further includes a third reference position sensor, and
the server receives third reference position information from the third reference position sensor, compares a first area calculated from the first reference position information, the third reference position information, and the first motion position information to a second area calculated from the second reference position information, the third reference position information, and the second motion position information, and generates the fitness correction information.

4. The real-time fitness coaching system of claim 1, wherein the smart fitness equipment further includes an identification (ID) module including type information of fitness equipment, and
the server includes a database for a plurality of pieces of fitness equipment for providing the fitness correction information, calls the database corresponding to the smart fitness equipment according to the type information of the fitness equipment received from the ID module, and generates the fitness correction information.

5. The real-time fitness coaching system of claim 4, wherein the smart wearable band further includes a pressure sensor, and
the server generates the fitness correction information on the basis of pressure information received from the pressure sensor.

6. The real-time fitness coaching system of claim 4, wherein the smart wearable band further includes a feedback module that provides feedback to a user in a preset manner, and
the smart wearable band receives the fitness correction information from the user terminal, and the feedback module provides the feedback to the user on the basis of the fitness correction information.

7. The real-time fitness coaching system of claim 6, wherein:
the feedback module is a vibration module; and
the feedback is a vibration pattern generated by the vibration module.

8. The real-time fitness coaching system of claim 6, wherein:
the feedback module is a speaker module; and
the feedback is a sound pattern or a guidance message generated by the speaker module.

9. The real-time fitness coaching system of claim 4, wherein the server provides a fitness motion to the user terminal as virtual image information on the basis of the reference position information and the motion position information.

10. The real-time fitness coaching system of claim 9, wherein the server adds a correction trace corresponding to the fitness correction information to the virtual image information and provides the virtual image information to the user terminal.
